(19) United States
(12) Patent Application Publication
(10) Pub. No.: US 2025/0025441 A1
(43) Pub. Date: Jan. 23, 2025

(54) PHARMACEUTICAL COMPOSITION AND A PROCESS TO PREPARE THE SAME

(71) Applicant: GODAVARI BIOREFINERIES LIMITED, Mumbai, Maharashtra (IN)

(72) Inventors: Vijayendra REDASANI, Maharashtra (IN); Sandip GAVADE, Mumbai, Maharashtra (IN); Maithili ATHAVALE, Mumbai, Maharashtra (IN); Prashant KHARKAR, Mumbai, Maharashtra (IN)

(21) Appl. No.: 18/692,298

(22) PCT Filed: Sep. 21, 2022
(86) PCT No.: PCT/IN2022/050842

§ 371 Date: Mar. 14, 2024

(30) Foreign Application Priority Data
Sep. 22, 2021 (IN) 202121042945

(51) Int. Cl. A61K 31/357; A61K 9/20; A61K 9/28; A61K 31/4025; A61K 31/7048

(52) U.S. Cl. CPC A61K 31/357; A61K 9/2027; A61K 9/2054; A61K 9/2095; A61K 9/282; A61K 31/4025; A61K 31/7048

(57) ABSTRACT

The present invention relates to pharmaceutical compositions comprising compound of Formula IV, along with a solubilizing agent and a process to prepare the same. The said pharmaceutical compositions have enormous applications in cancer therapy. The present invention has come up with a novel and innovative pharmaceutical composition for the compounds of Formula IV that show significant stability as well as the desired bioavailability on drug release.

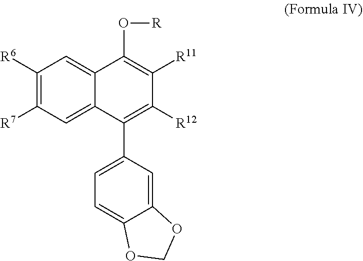

### FIELD OF THE INVENTION

The present invention relates to a pharmaceutical composition comprising compound of Formula IV, along with a solubilizing agent and a process to prepare the same. The said pharmaceutical composition has enormous applications in cancer therapy

### BACKGROUND OF THE INVENTION

Solubility, the phenomenon of dissolution of solute in solvent to give a homogenous system, is one of the important parameters to achieve desired concentration of drug in systemic circulation for desired pharmacological response. Low aqueous solubility is the major problem encountered with formulation development of new chemical entities as well as for the generic development. More than 40% NCEs (new chemical entities) developed in pharmaceutical industry are practically insoluble in water. The poor solubility and low dissolution rate of poorly water-soluble drugs in the aqueous gastrointestinal fluids cause insufficient bioavailability. The negative effect of compounds with low solubility includes poor absorption and bioavailability, insufficient solubility for IV dosing, development challenges leading to increasing the development cost and time, burden shifted to patient. (10.5402/2012/195727).

Various techniques are used for the enhancement of the solubility of poorly soluble drugs which include physical and chemical modifications of drug and other methods like particle size reduction, crystal engineering, salt formation, solid dispersion, use of surfactant, complexation, and so forth. Selection of solubility improving method depends on drug property, site of absorption, and required dosage form characteristics.

Solubilizing agents, such as surfactants, are solubilizer excipients added to pharmaceutical formulations in order to increase the solubility of poorly soluble drugs, thereby increasing the bioavailability of the corresponding active pharmaceutical ingredient (API). However, it is found that the solubility of poorly water-soluble drugs is way below the desired threshold, which lowers their absorption in the gastrointestinal tract. Also, their stability is lowered considerably. Furthermore, it is well known that poorly water-soluble drugs have reduced drug efficiencies as compared to pharmaceutical formulations with appropriate solubility profiles and may cause undesirable side effects upon administration.

In the BCS (Biopharmaceutics Classification System) classification all drugs have been divided into four classes: class I—high soluble and high permeable, class II—low soluble and high permeable, class III—low soluble and high permeable and class IV—low soluble and low permeable. BCS class II drugs have high absorption number and low dissolution number. The absorption/bioavailability is limited by dissolution rate.

The inventors in the present invention have focused on improving the dissolution of the compounds of Formula IV. The compounds of Formula IV are compounds that demonstrate anticancer and anti-cancer stem cell activity particularly in breast and prostate cancer cell lines. Further, the compounds have also found to be having antiviral property.

The inventors appreciate that the stability of finished pharmaceutical products is also very important. It depends on one hand, on environmental factors such as ambient temperature, humidity and light, and, on the other, on product-related factors, e.g. the chemical and physical properties of the active substance and of pharmaceutical excipients, the dosage form and its composition, the manufacturing process, the nature of the container-closure system and the properties of the packaging materials.

It is a constant challenge to produce a drug product which shows good stability and at the same time has a good dissolution profile which would result in high bioavailability. The present invention addresses this challenge for pharmaceuticals and has come up with a novel and innovative pharmaceutical composition for the compounds of Formula IV that show significant stability as well as the desired bioavailability on drug release.

### OBJECTS OF THE INVENTION

The object of the invention is to improve the solubility of the compound of Formula IV in a suitable formulation or in a drug product.

Another object of the invention is to improve the stability of the compounds of Formula IV at accelerated conditions.

Another object of the invention is to improve the stability of the compounds of Formula IV in a packed form without degradation of its amorphous form.

Yet another object of the invention is to improve the bioavailability of the compounds of Formula IV.

It is another object of the invention to provide for compositions of the compounds of Formula IV which are highly soluble in physiological buffers.

Another object of the invention is to provide for a pharmaceutical composition of the compounds of Formula IV for the treatment of cancer, especially breast cancer.

Yet another object of the invention is to provide for a solid oral unit dose formulation of the compounds of Formula IV.

Yet another object of the invention is to provide for tablet dosage forms with high drug release of compounds of Formula IV.

It is yet another object of the invention to provide for an environmentally friendly method of preparing pharmaceutical compositions of the compounds of Formula IV.

### SUMMARY OF THE INVENTION

The present invention is directed towards a pharmaceutical composition comprising the compound of Formula IV or its pharmaceutically acceptable salt thereof,

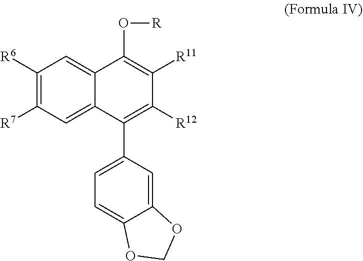

wherein R⁶ and R⁷ each independently is selected from —H, alkoxy, alkyl, substituted or unsubstituted aromatic group, —NH₂, —NO₂, —NHCOCH₃, —CN, —O—, halogen, —OCF₃ or R⁶ and R⁷ together form a heterocyclic ring;
R¹¹ and R¹² each independently is selected from —H, or R¹¹ and R¹² can be substituted or unsubstituted 5- or 6-membered ring such as lactone, —C(O)O-alkyl such as —C(O)OC₂H₅; R is selected from

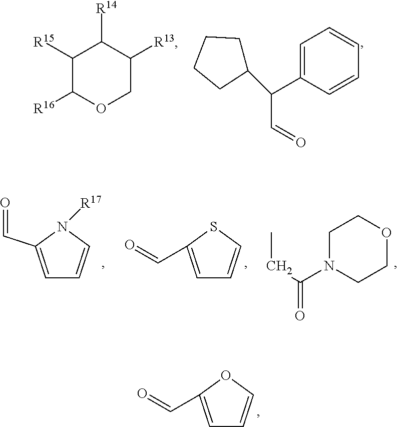

—H, —C(O)CH₂Cl, —SOO—CH₃, —SOOPh, —CH₂C(O)N(CH₃)₂, —C(O)NHPh, —C(O)NHPhOH, —C(S)NHPh, —CH₂Ph, —COAr, —SOOAr, —CONHAr, —CH₂Ar, —CSNHAr, wherein, R¹³ is selected from —OH, —NH₂, —NHCOCH₃, X═F, Cl, Br, alkyl, acetyl, C₃-C₈ acyl group; R¹⁴ is selected from alkoxy, —OMe, —OH, NH₂, —NHCOCH₃, X═F, Cl, Br, alkyl, acetyl, C₃-C₈ acyl group; R¹⁵ is selected from alkoxy, —OMe, —OH, —H, Br, NH₂, X═F, Cl, Br, alkyl, acetyl, C₃-C₈ acyl group; R¹⁶ is selected from —H, —CH₂OH, —OH, alkyl, alkoxy; R¹⁷ is selected from alkyl; along with a solubilizing agent, further wherein the compound or a pharmaceutically acceptable salt of Formula IV, in the said pharmaceutical composition is amorphous.

The claimed pharmaceutical composition has improved solubility and drug release for the compounds of Formula IV and at the same time has a very good stability in packed form profile extending up to 18 months not undergoing any degradation in its amorphous form.

The pharmaceutical composition is in a dosage form, preferably in the form of a tablet for oral administration.

### DETAILED DESCRIPTION OF THE INVENTION

The present invention relates to a pharmaceutical composition comprising any of the compound of Formula IV;

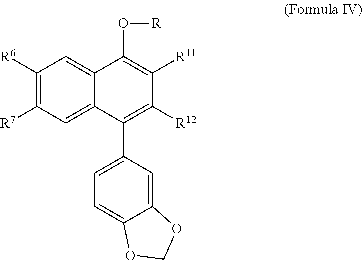

wherein R⁶ and R⁷ each independently is selected from —H, alkoxy, alkyl, substituted or unsubstituted aromatic group, —NH₂, —NO₂, —NHCOCH₃, —CN, —O—, halogen, —OCF₃ or R⁶ and R⁷ together form a heterocyclic ring;
R¹¹ and R¹² each independently is selected from —H, or R¹¹ and R¹² can be substituted or unsubstituted 5- or 6-membered ring such as lactone, —C(O)O-alkyl such as —C(O)OC₂H₅; R is selected from

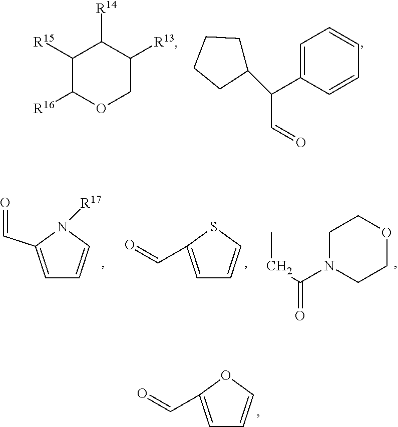

—H, —C(O)CH₂C₁, —SOO—CH₃, —SOOPh, —CH₂C(O)N(CH₃)₂, —C(O)NHPh, —C(O)NHPhOH, —C(S)NHPh, —CH₂Ph, —COAr, —SOOAr, —CONHAr, —CH₂Ar, —CSNHAr, wherein, R¹³ is selected from —OH, —NH₂, —NHCOCH₃, X═F, Cl, Br, alkyl, acetyl, C₃-C₈ acyl group; R¹⁴ is selected from alkoxy, —OMe, —OH, NH₂, —NHCOCH₃, X═F, Cl, Br, alkyl, acetyl, C₃-C₈ acyl group; R¹⁵ is selected from alkoxy, —OMe, —OH, —H, Br, NH₂, X═F, Cl, Br, alkyl, acetyl, C₃-C₈ acyl group; R¹⁶ is selected from —H, —CH₂OH, —OH, alkyl, alkoxy; R¹⁷ is selected from alkyl; along with a solubilizing agent, further wherein the compound or a pharmaceutically acceptable salt of any of the compound of Formula IV, in the said pharmaceutical composition is amorphous.

The pharmaceutical compositions are highly stable in a packed form and do not degrade with time. The claimed compositions also shows significant solubility in physiological buffers.

The compounds of the present invention showed poor solubility as currently available in the market. The solubilization capacity of drug compositions are measured in aqueous media which is a primary property of a drug substance. However, it is also critical to understand the solubility in buffer solutions that mimic conditions inside the body such as the gastrointestinal tract, particularly for drugs which are lipophilic and sparingly soluble. The presently claimed composition are surprisingly soluble in multiple buffer solutions. Also, the claimed compositions show excellent stability in accelerated condition. The compositions as claimed further show excellent stability upto 18 months in a packed form and do not degrade with time.

In a preferred embodiment, the compounds of Formula IV are selected from:

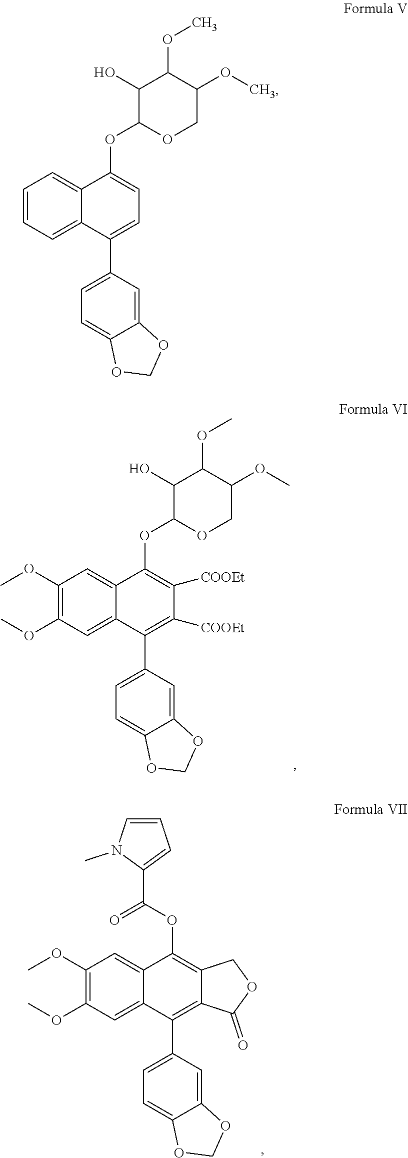

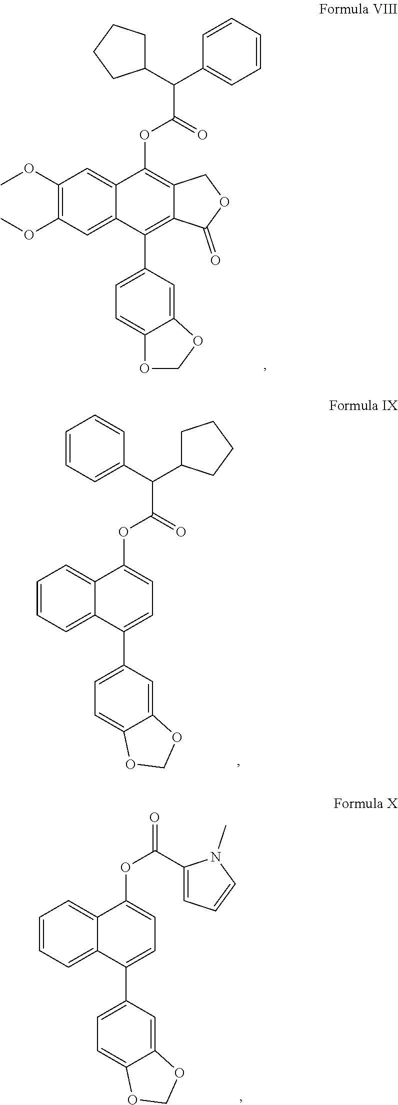 [SMILES: COc1cc2c(OC(=O)C(c3ccccc3)C3CCCC3)c3c(c(-c4ccc5c(c4)OCO5)c2cc1OC)C(=O)OC3 ; O=C(Oc1ccc(-c2ccc3c(c2)OCO3)c2ccccc12)C(c1ccccc1)C1CCCC1 ; Cn1cccc1C(=O)Oc1ccc(-c2ccc3c(c2)OCO3)c2ccccc12]

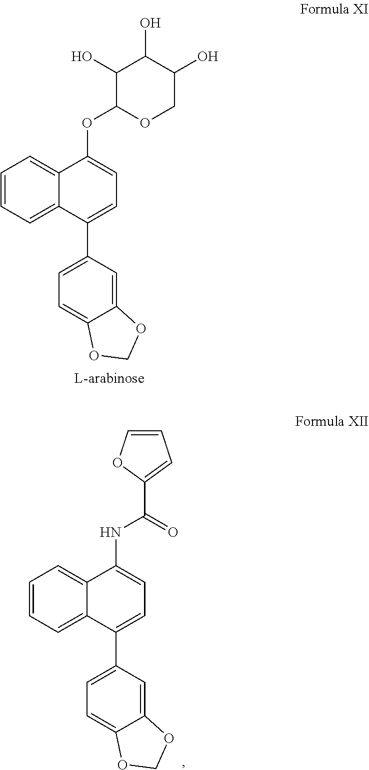 [SMILES: OC1COC(Oc2ccc(-c3ccc4c(c3)OCO4)c3ccccc23)C(O)C1O ; O=C(Nc1ccc(-c2ccc3c(c2)OCO3)c2ccccc12)c1ccco1]

The present invention achieves a drug composition wherein the solubility of the compounds of Formula IV is enhanced in such a manner that it results in increased drug release, and bioavailability. The invention also provides a highly stable composition in accelerated conditions, which simultaneously also achieves enhanced solubility and bioavailability of the compounds of Formula IV.

Therefore, the composition was found to have an improved dissolution profile. The presently claimed composition show good solubility not only in aqueous media but are surprisingly soluble in multiple physiological buffer solutions and at the same time are also stable upto 18 months in a packed form and even in accelerated conditions.

### Definitions

Unless defined otherwise, all technical and scientific terms used herein have the same meaning as commonly understood by one of ordinary skill in the art to which this invention belongs.

Although any methods and materials similar or equivalent to those described herein can be used in the practice or testing of the present invention, the preferred methods and materials are described herein. As used herein, each of the following terms has the meaning associated with it in this section. Specific and preferred values listed below for individual process parameters, substituents, and ranges are for illustration only; they do not exclude other defined values or other values falling within the preferred defined ranges.

The term amorphous as used herein refers to relates to solids having short range order but not having long range order as in crystalline substances.

PVPK-30 as used herein refers to polyvinyl pyrrolidone K30

Tween 30/20 as used herein refers to relate to polysorbates

Kollidone VA 64 as used herein refers to relate to vinylpyrrolidone and vinyl acetate copolymer.

Kolliphor SLS as used herein refers to relates to an alkyl sulphate on the basis of a natural, saturated, straight-chain, primary fatty alcohol.

HPBCD as used herein refers to 2-Hydroxypropyl-β-cyclodextrin (HP-β-CD)

CRYSMEB as used herein refers to low methylated beta cyclodextrin product

Avicel SMCC 90 as used herein refers to a high functionality co-processed spray-dried excipient comprising of 98 percent of microcrystalline cellulose (MCC) and 2 percent of colloidal silicon dioxide.

Cross carmellose sodium as used herein refers to internally cross-linked sodium carboxymethylcellulose

Aerosil 200 as used herein refers to hydrophilic fumed silica, Methocel E-15 LV as used herein refers to low molecular weight hydroxypropyl methylcellulose (HPMC)

Physiological pH as used herein relates to the pH of a human body.

Physiological Buffer used herein refers to buffer systems present in the body that prevent sudden and rapid changes in the pH

The phrase “stability in a packed form” used herein refers to stability of drug compositions in a suitable formulation relating to their shelf life in a form as initially prepared which has high solubility and bioavailability.

The phrase “accelerated conditions” refers to condition of elevated temperature (40° C.) at 75% relative humidity. Accelerated stability testing Studies are designed to increase the rate of chemical degradation and physical change of a drug by using exaggerated storage conditions as part of the formal stability testing programme guided by ICH (International Council for Harmonisation of Technical Requirements for Pharmaceuticals for Human Use).

Vitamin E TPGS as used herein refers to TPGS is the polyethylene glycol 1000 ester of d-α-tocopheryl succinate. It is chemically stable to heat, oxygen and light is used as solubility enhancer.

The pharmaceutical composition of the present invention comprises the compound of the Formula IV in a range of 10% to 95%, preferably 40% to 95%, more preferably 50% to 95% by weight of the composition.

The pharmaceutical compounds of the present invention comprise of one or more solubilizing agent in the range of 2% to 20% by weight of the composition, more preferably in the range of 4% to 10% by weight of the composition.

Solubilizing agents are selected from polyvinylpyrrolidone (povidone), cyclodextrin, derivative of cyclodextrin, Tween 80, Tween 20, PVPK-30, Citric acid, Kollidone VA64, Kolliphor SLS, Polyethylene glycol or mixtures, thereof.

Preferably, the solubilizing agent is povidone specifically polyvinyl pyrrolidone and cyclodextrin specifically HPBCD (hydroxypropyl beta cyclodextrin). The solubilizing agent may also be selected from sulfobutylether-cyclodextrin or CRYSMEB-Low methylated beta cyclodextrin.

Preferably, the amount of solubilizing agent in the pharmaceutical composition is in a range of 2 to 20% by weight, preferably 4 to 10% by weight.

Preferably, the pharmaceutical composition in accordance with this invention can be administered in a variety of ways.

The pharmaceutical composition containing a compound of Formula IV can be administered in therapeutically effective amounts as pharmaceutical compositions by any conventional form and route known in the art including, but not limited to: intravenous, intra-arterial, intramuscular, subcutaneous, oral, rectal, aerosol, parenteral, ophthalmic, pulmonary, transdermal, vaginal, nasal, and topical administration.

The pharmaceutical composition comprising the compounds of Formula IV can be administered by way of solid dose forms including dissolvable tablets, granule, gums, lozenges, pellets, and other forms for intraoral delivery by sublingual and buccal administration. Preferably, the claimed pharmaceutical composition is to be administered by way of oral administration, preferably in a tablet formulation.

In another embodiment, the present invention relates to a process to prepare pharmaceutical composition comprising the steps of mixing compound of formula IV and solubilizing agent. Preferably, hot melt extrusion technique, wet granulation process, dry granulation process or direct compression is used for preparing the composition. More preferably, hot melt extrusion technique is used for preparing the composition.

### Preparation:

### Process I (Hot Melt Extrusion)

The hot melt extrusion technique is an environment friendly process as it is solvent free, resulting in less effluent generation. Preferably, the solubilizing agent is polyvinylpyrrolidone when the process of preparation is hot melt extrusion.

The process includes

Dispensing: Maintaining the humidity in the dispensing area to be not more than 50% RH. Calibrating all the balances before dispensing. Sifting: Co-sifting the compound of Formula IV and solubilizing agent, preferably polyvinylpyrrolidone through ASTM mesh #40. Collecting the mixture and keeping it aside for further processing. Dry Blending: Blending the mixture after co-sifting in a suitable capacity blender for 10 minutes at 4 to 40 RPM (revolutions per minute), preferably 10 to 20 RPM. Extrusion Process: Passing the blended mixture through gravity feeder in twin screw barrel of suitable extrusion. Setting the parameters of zones in twin screw barrel and starting extrusion process. Then, setting suitable RPM (revolutions per minute) of feeder and screw so that the torque not be more than 50%. Checking the extruded mixture for consistency and clarity. Passing the extruded mixture through Co-mill/multi mill with 0.8 mm screen and followed by through ASTM mesh #40. Repeating the process till all extruded mixture pass through ASTM #40 mesh. Sifting: Co-sifting the powder extruded mixture along with desired quantity of extragranular ingredients selected from Lactose Monohydrate (Granulac® 200), Silicified Microcrystalline cellulose (Avicel SMCC 90), Colloidal Silicon dioxide (Aerosil 200) or combination thereof through ASTM #40 mesh. Blending:Mixing the above mixture in suitable capacity blender for 10 min at 16 RPM. Lubrication:Sifting lubricants such as magnesium stearate through ASTM mesh #60 and adding to the blended mixture of step 6. Mixing and lubricating the above blended mixture for 5 minutes at 16 RPM. Unloading the lubricated blend in double polythene bag lined container.

Extragranular ingredients is selected from lactose monohydrate (Granulac® 200), silicified microcrystalline cellulose (Avicel SMCC 90), cross carmellose sodium (Ac Di Sol SD 711), colloidal silicon dioxide (Aerosil 200), hydroxypropyl methyl cellulose (Methocel E-15 LV Premium) or combination thereof. The amount of extragranular ingredients is the composition is in a range of 0.2 to 40% by weight, preferably 0.2 to 20% by weight, more preferably 0.2 to 11% by weight.

Intragranular ingredients are selected from lactose monohydrate (Granulac® 200), silicified microcrystalline cellulose (Avicel SMCC 90), cross carmellose sodium (Ac Di Sol SD 711), Hydroxyl Beta cyclodextrin (HPBCD), polysorbate 80, colloidal silicon dioxide (Aerosil 200), hydroxypropyl methyl cellulose (Methocel E-15 LV Premium) or combination thereof. The amount of intragranular ingredients is the composition is in a range of 0.3 to 14% by weight.

Lubricant is selected from magnesium stearate, sodium stearyl fumarate, stearic acid, colloidal silicon dioxide etc. The amount of lubricant in the composition is in a range of 0.4 to 0.5% by weight.

Polymer is selected from Copovidone, polyvinylpolypyrrolidone (PVP), hydroxypropylcellulose (HPC), polyethylene glycol (PEG), hydroxypropyl methyl cellulose (HPMC), hypromellose acetate succinate (HPMCAS), Copovidone/vitamin E/Polyethylene glycol, Copovidone/Polysorbate 80/Colloidal silicon dioxide.

### BRIEF DESCRIPTION OF DRAWINGS

FIG. 1: The claimed pharmaceutical composition shows superior % drug release over a period of 60 minutes. This correlates with the tabulated data of Experiment 2

FIG. 2: The claimed pharmaceutical composition shows superior % drug release over a period of 60 minutes. This correlates with the tabulated data of Experiment 3

FIG. 3: The claimed pharmaceutical composition shows superior % drug release over a period of 60 minutes. This correlates with the tabulated data of Experiment 4 wherein different comparative compositions comprising the compound of Formula IV have been taken.

FIG. 4: The absence of endothermic peak at 145 Deg (Melting Point of Formula V as per present invention) suggest that the Crystalline form is converted to Amorphous form in the as claimed pharmaceutical composition.

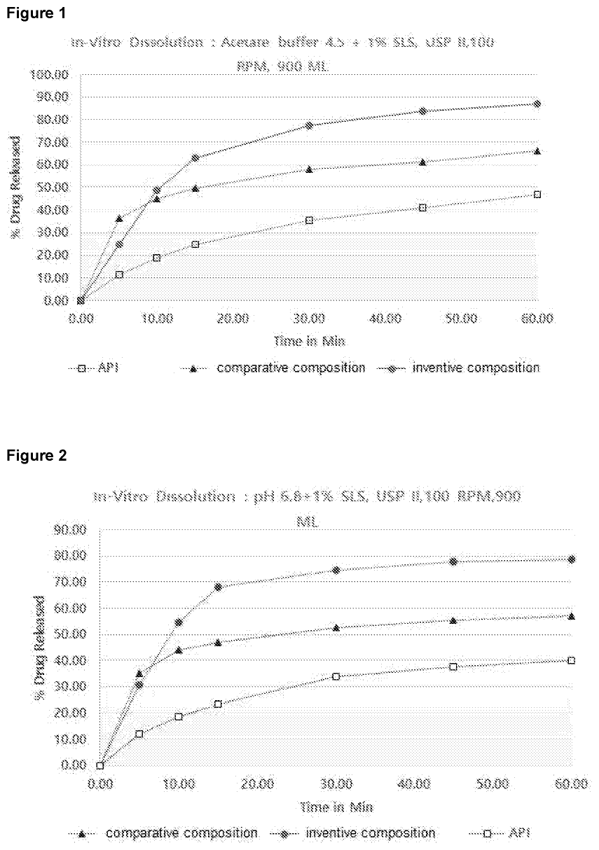

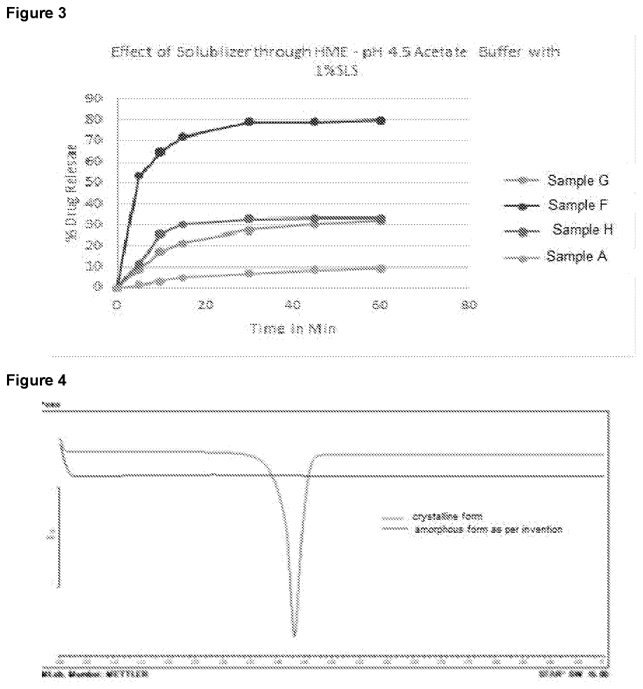

### EMBODIMENTS

The present invention envisages an embodiment of the present invention wherein the crystalline Form of compound IV is converted to the amorphous form to make the drug more bioavailable.

In an embodiment of the present invention, the pharmaceutically acceptable salts of the compounds of Formula IV along with a solubilizing agent, form part of the claimed pharmaceutical composition

In a preferred embodiment, the compound of Formula IV is Formula V.

An embodiment of the present invention wherein the crystalline Form of compound V is converted to amorphous form to make the drug more bioavailable.

In an embodiment of the present invention the pharmaceutical composition of the present invention comprises the compound of the Formula IV in a range of 10% to 95%, preferably 40% to 95%, more preferably 50% to 95% by weight of the composition.

In another embodiment of the present invention the pharmaceutical composition of the present invention comprises the compound of the Formula IV in a range of 40% to 90%, preferably 30% to 90%, more preferably 50% to 90% by weight of the composition.

In an embodiment of the present invention of the pharmaceutical compounds of the present invention comprise of one or more solubilizing agents in the range of 2% to 20% by weight of the composition, more preferably in the range of 4% to 10% by weight of the composition.

### Process II (Wet Granulation Process)

The pharmaceutical composition of the present invention may also be arrived at by the Wet granulation Process. Preferably, the solubilizing agent is cyclodextrin, in this method.

The method comprises of the following steps:

Dispensing: Weighing compound of Formula IV along with pharmaceutically acceptable excipients. Passing intragranular ingredients through ASTM mesh #30. Preparing binder solution: Adding required quantity of water and Polysorbate 80 to the above mixture and stirring the solution and adding the cyclodextrin under continuous stirring to the above blend. The method further comprises preparing the tablet from the blend. The method comprises granulating, drying, blending, lubricating and compressing the above blend. Granulation is selected from steam granulation, moist granulation, melt granulation, hand granulation. Preferably granulation is hand granulation. Granules are dried in hot air oven for 60° C. for 60 min. After drying granules are passed through mesh #30 followed by adding the extragranular ingredient selected from Cross Carmellose Sodium (Ac Di Sol SD 711), Silicified Microcrystalline cellulose (Avicel SMCC 90), Colloidal Silicon dioxide (Aerosil 200) or combination thereof and pass through mesh #30 and mix for 10 min. Sifting the lubricant such as magnesium stearate through ASTM mesh 60 # and adding the sifted magnesium stearate into the above mixture for 5 min. Compression: In an embodiment, the lubricated blend obtained from process I and/or process II were further compressed to form tablets. The process of compression is carried out using a compression machine. Coating: In an embodiment, the tablet is coated using a coating agent. Coating of the tablets can be film coating, enteric coating or delayed release coating. Film coating agent is formed from a coating premix (Opadry II Purple 85F90093), sodium lauryl sulphate (Kolliphor SLS Fine) hydroxypropylmethylcellulose (HPMC) or polyvinyl alcohol (PVA) based or combination thereof.

The binder is selected from hydroxypropyl betadex, hydroxypropyl beta cyclodextrin (KleptoseHPB), polysorbate 80, purified water or combination thereof. The amount of binder in the composition is in a range of 2% to q.s. by weight. Tablet binder or binding agent are the substances which are added either dry or in liquid form during wet granulation to form granules or to promote cohesive compacts for directly compressed tablets.

### Formulation of Tablets—Coating Process

Taking 20% of total quantity of Purified Water in the stainless-steel vessel. Dissolving weighed quantity of sodium lauryl sulphate (Kolliphor SLS Fine) under slow and continuous stirring. Adding weighed quantity of Opadry II Purple 85F90093 to the above solution of sodium lauryl sulphate, slowly under continuous stirring and stirring for 45 minutes in order to avoid formation of foam. Filtering the above solution/coating dispersion through ASTM mesh #40 and keeping dispersion under slow continuous stirring throughout process. Loading the tablets of compound of formula IV to the coating machine to achieve the weight gain of 4.00±0.2% w/w. Pre-warming the tablets to achieve the bed temperature 35° C. to 45° C. by rotating pan at 1-2 RPM (revolutions per minute). After getting the desired bed temperature, coating the tablet by maintaining the above-mentioned suitable parameters. After appropriate desired weight gain is achieved, drying tablets at bed temperature of not more than (NMT) 50° C., with pan at 1-2 RPM, for 15 minutes. Cooling the tablets and unloading in double polythene lined container and storing in HDPE container/IPC with silica gel as desiccant.

The present invention also envisages a method of treating cancer and cancer related disorders in an individual, comprising administering to the individual an effective amount of the pharmaceutical composition comprising the compounds of the Formula IV along with a solubilizing agent.

### Working Examples

The inventors by way of experimentation have sought to arrive at the most viable pharmaceutical composition of the compounds of formula IV, which achieves significantly enhanced drug release.

EXPERIMENT 1
To study the effect of different physiological pH Medium on
dissolution of the compounds of Formula IV

| | In-vitro Dissolution: USP Type II Apparatus (Paddle), | | | | |
|---|---|---|---|---|---|
| | 50 RPM, 500 ml | | | | |
| | | | pH 6.8 | pH 4.5 | pH 7.4 |
| Dissolution | Purified | 0.1N | Phosphate | Acetate | Phosphate |
| Condition | Water | HCl | Buffer | Buffer | Buffer |
| Strengths | 200.00 | 200.00 | 200.00 | 200.00 | 200.00 |
| Time in | % Drug | % Drug | % Drug | % Drug | % Drug |
| Min | Release | Release | Release | Release | Release |
| 0.00 | 0.00 | 0.00 | 0.00 | 0.00 | 0.00 |
| 5.00 | 0.00 | 0.00 | 0.00 | 0.00 | 0.00 |
| 10.00 | 0.00 | 0.00 | 0.00 | 0.00 | 0.00 |
| 15.00 | 0.00 | 0.00 | 0.00 | 0.00 | 0.00 |
| 30.00 | 0.00 | 0.00 | 0.00 | 0.00 | 0.00 |
| 45.00 | 0.10 | 0.10 | 0.00 | 0.10 | 0.10 |
| 60.00 | 0.00 | 0.00 | 0.00 | 0.00 | 0.00 |
| REC | 0.10 | 0.00 | 0.10 | 0.01 | 0.00 |

The data indicates that the compounds of Formula IV without the claimed pharmaceutical composition have no solubility in different physiological pH medium.

A study of the dissolution profile of the claimed pharmaceutical was conducted in different physiological buffers and compared to the dissolution profile of the API and a pharmaceutical composition without the inventive composition in Experiments 2 and 3.

EXPERIMENT 2
The dissolution condition in this experiment was acetate buffer
pH 4.5 + 1% SLS, USP II, 1000 RPM, 900 ML

| | | Composition as per | Comparative |
|---|---|---|---|
| Time in | | invention | composition |
| minutes | API | With VA 64 | without VA 64 |
| 0 | 0.00 | 0.00 | 0.00 |
| 5 | 11.30 | 24.90 | 36.60 |
| 10 | 19.10 | 48.70 | 45.20 |
| 15 | 25.00 | 63.00 | 49.80 |
| 30 | 35.50 | 77.40 | 57.90 |
| 45 | 41.20 | 83.90 | 61.20 |
| 60 | 47.00 | 87.30 | 66.40 |

From the above comparison, it is observed that there is significant increase in the drug release in the claimed composition as compared to the API.

EXPERIMENT 3:
The dissolution condition in this experiment was acetate
pH6.8 + 1% SLS, USP II, 1000 RPM, 900 ML

| | | Composition as per | Comparative |
|---|---|---|---|
| Time in | | invention | composition |
| minutes | API | With VA 64 | without VA 64 |
| 0 | 0.00 | 0.00 | 0.00 |
| 5 | 12.20 | 30.70 | 35.30 |
| 10 | 18.40 | 54.80 | 44.10 |
| 15 | 23.60 | 68.30 | 46.90 |
| 30 | 33.80 | 74.80 | 52.80 |
| 45 | 37.80 | 78.00 | 55.40 |
| 60 | 40.00 | 78.80 | 57.00 |

From the above comparison, it is observed that there is significant increase in the drug release in the claimed composition as compared to the API.

EXPERIMENT 4
In experiment 4, drug release for different pharmaceutical compositions of the compounds of Formula
IV were studied in acetate buffer with 1% SLS (sodium lauryl sulphate). The results are produced under:

| | Composition | | | | | | | |
|---|---|---|---|---|---|---|---|---|
| | | | VA64(2X) + | | VA64 + | VA64 | | |
| | API | SLS | TPGS | SLS | TPGS + SLS | (1:2) | HPMCAS | PVA |
| | Process | | | | | | | |
| | | | Wet | Wet | Wet | | | |
| | Hot melt | Wet granulation | granulation | granulation | granulation | Hot melt | Hot melt | Hot melt |
| | extrusion | Water + PVPK30 | acetone | acetone | acetone | extrusion | extrusion | extrusion |
| | Sample | | | | | | | |
| Time in min | | | | | | F (as per | | |
| For % drug | | | | | | present | | |
| release | A | B | C | D | E | invention) | G | H |
| 0 | 0 | 0 | 0 | 0 | 0 | 0 | 0 | 0 |
| 5 | 1.3 | 37 | 16 | 34 | 15 | 54 | 9 | 12 |
| 10 | 3.4 | 45 | 36 | 48 | 29 | 65 | 17 | 26 |
| 15 | 4.8 | 50 | 53 | 55 | 42 | 72 | 21 | 30 |
| 30 | 6.9 | 58 | 65 | 66 | 62 | 79 | 27 | 33 |
| 45 | 8.5 | 61 | 66 | 69 | 70 | 79 | 30 | 33 |
| 60 | 9.4 | 66 | 73 | 75 | 73 | 80 | 32 | 33 |

The above results are indicative of the enhanced efficacy of the claimed composition. The pharmaceutical composition as per the present invention shows tremendous improvement in drug release from the beginning at 5, 10, 15, 30, 45 and 60 minutes.

When the compound (API) is without a solubilizing agent, then the drug release is minimal. When the composition comprises the active compound along with a copolymer of vinylpyrrolidone and vinyl acetate, produced via the wet granulation, the drug release shows some improvement, but it is not sufficient to have significant effect on the final dosage. When the composition comprises SLS, it is again noted that though drug release has improved, it is similar to what is obtained when the composition is produced by wet granulation.

It can be concluded that when the composition comprises the active compound along with VA 64 in the desired wt % as per the present invention (sample F), produced by hot melt extrusion, the inventors are surprisingly achieving major improvement in the % drug release.

Experiment 5: Accelerated, intermediate and long term stability tested for Compound V in the claimed pharmaceutical composition.

| | | | Condition | | | | | 25° C./ |
|---|---|---|---|---|---|---|---|---|
| | | | | 40° C./75% RH | | | 30° C./ | 60% RH |
| | | | | 1 | 2 | 3 | 75% RH | upto 3 |
| Sr. No. | Test | Specifications | Initial | Month | Months | Months | 3 Months | Months |
| 1 | Description | Light peach to purple | Complies | Complies | Complies | Complies | Complies | Complies |
| | | colured, oval-shaped, | | | | | | |
| | | biconvex film-coated | | | | | | |
| | | tablets, plain on | | | | | | |
| | | other side. | | | | | | |
| 2 | Assay by HPLC | Each tablet contain | 101.1% | 103.3% | 99.1% | 98.8% | 99.9% | 99.9% |
| | | 90.0% of Compound | | | | | | |
| | | of Formula V | | | | | | |
| 3 | Dissolution by HPLC | Not less than 70%(Q) | 93.40% | 93.30% | 96.10% | 90.90% | 97.90% | 95.20% |
| | Apparatus: USP Type-II, | of the labeled amount | | | | | | |
| | Dissolution medium: 2.0% | of Compound V is | | | | | | |
| | SLS in pH 4.5 Acetate | dissolved in 60 | | | | | | |
| | buffer, Volume: 900 mL | minutes. | | | | | | |
| | RPM: 100, | | | | | | | |
| | Temperature: 37° C.(±0.5° C.) | | | | | | | |

The results show that the claimed pharmaceutical compositions have significant stability in accelerated conditions. Also, the pharmaceutical composition as claimed, comprises the compound in the amorphous form which does not degrade with time and retains its amorphous character. It is well understood by a skilled person in art that the stability of the composition of the present invention in view of the above data is extendable upto 18 months, in packed form.

TABLE 9
Experiment 6: Stability study:
Stability of the pharmaceutical composition in acetate buffer
of 4.5 + 2% SLS, USP II, 100 RPM, 900 ML for
1 month, 2 months and 3 months at different
time i.e., at 0 hrs, 5 hrs, 10 hrs, 15 hrs, 30 hrs, 45 hrs and
60 hrs, respectively was studied and is represented below:

| | Acetate buffer | Acetate buffer | Acetate buffer |
|---|---|---|---|
| | 4.5 + 2% SLS, | 4.5 + 2% SLS, | 4.5 + 2% |
| | USP | USP | SLS, USP |
| Dissolution | II, 100 RPM, | II, 100 RPM, | II, 100 RPM, |
| Condition | 900 ML | 900 ML | 900 ML |
| Time in Min/ | 1M | 2M | 3M 40° C./ |
| Condition | 40° C./75% RH | 40° C./75% RH | 75% RH |
| 0.00 | 0.00 | 0.00 | 0.00 |
| 5.00 | 69.10 | 72.50 | 68.10 |
| 10.00 | 77.40 | 83.70 | 75.90 |
| 15.00 | 82.60 | 88.00 | 80.80 |
| 30.00 | 88.60 | 92.40 | 88.40 |
| 45.00 | 91.90 | 94.70 | 90.30 |
| 60.00 | 93.30 | 96.10 | 90.90 |

The dissolution data on stability infers that the claimed composition is stable at accelerated condition with no significant change in drug release.

### APPLICATION

The pharmaceutical composition can be used for treatment of cancer of the breast, oral, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, or stomach; preferably for breast cancer and cancer related diseases.

1. A pharmaceutical composition comprising the compound of Formula IV or its pharmaceutically acceptable salt thereof, 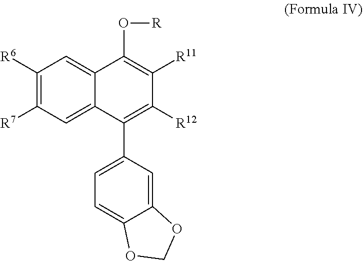
wherein R6 and R7 each independently is selected from —H, alkoxy, alkyl, substituted or unsubstituted aromatic group, —NH2, —NO2, —NHCOCH3, —CN, —O—, halogen, —OCF3 or R6 and R7 together form a heterocyclic ring;
R11 and R12 each independently is selected from —H, or R11 and R12 can be substituted or unsubstituted 5- or 6-membered ring such as lactone, —C(O)O-alkyl such as —C(O)OC2H5; R is selected from 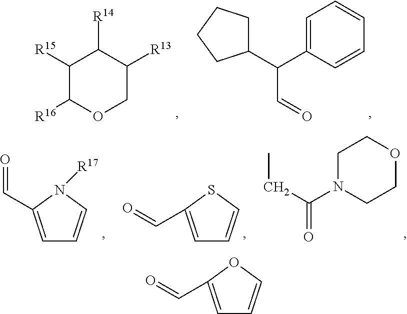
—H, —C(O)CH2Cl, —SOO—CH3, —SOOPh, —CH2C(O)N(CH3)2, —C(O)NHPh, —C(O)NHPhOH, —C(S)NHPh, —CH2Ph, —COAr, —SOOAr, —CONHAr, —CH₂Ar, —CSNHAr, wherein, R13 is selected from —OH, —NH2, —NHCOCH3, X═F, Cl, Br, alkyl, acetyl, C3-C8 acyl group; R14 is selected from alkoxy, —OMe, —OH, NH2, —NHCOCH3, X═F, Cl, Br, alkyl, acetyl, C3-C8 acyl group; R15 is selected from alkoxy, —OMe, —OH, —H, Br, NH2, X═F, Cl, Br, alkyl, acetyl, C3-C8 acyl group; R16 is selected from —H, —CH₂OH, —OH, alkyl, alkoxy, R17 is selected from alkyl;
and a solubilizing agent, further wherein the compound or a pharmaceutically acceptable salt of Formula IV, in the said pharmaceutical composition is amorphous.

2. The composition as claimed in claim 1, wherein the composition is stable at accelerated conditions.

3. The composition as claimed in claim 1, wherein the amount of compound of Formula IV, is in a range of 10% to 95% preferably 40% to 95%, more preferably 50% to 95% by weight of the composition.

4. The composition as claimed in claim 1, wherein the amount of solubilizing agent is in the range of 2% to 20% by weight of the composition, more preferably in the range of 4% to 10% by weight of the composition and is selected from polyvinylpyrrolidone, cyclodextrin, derivative of cyclodextrin, Tween 80, Tween 20, PVPK-30, citric acid, Kolidone VA64, Polyethylene glycol or mixtures thereof.

5. (canceled)

6. The composition as claimed in claim 1, wherein the solubilizing agent is either a cyclodextrin or a polyvinylpyrrolidone.

7. The composition as claimed in claim 1 optionally comprising components selected from film coating, extragranular ingredients, intragranular ingredients, binder, lubricant, stabilizer, glidant, surface active agent, diluents, disintegrants, pH adjusting agent, polymer or mixtures thereof.

8. The composition as claimed in claim 1, wherein the lubricant in the composition is in the range of 0.4% to 0.5% by weight of the composition and is selected from magnesium stearate, sodium stearyl fumarate, stearic acid and colloidal silicon dioxide.

9. The composition as claimed in claim 1, wherein the binder is in the range of 2% to q.s, y weight of the composition and is selected from hydroxypropyl betadex, hydroxypropyl betacyclodextrin, polysorbate 80, purified water or a combination thereof.

10. The composition as claimed in claim 1, wherein the extra granular ingredient is in the range of 0.2% to 40%, preferably 0.2% to 20%, more preferably 0.2% to 11% by weight of the composition and is selected from lactose monohydrate, silicified microcrystalline cellulose, cross carmellose sodium, colloidal silicon dioxide, hydroxypropyl methyl cellulose or a combination thereof.

11. The composition as claimed in claim 1, wherein the intra granular ingredient is in the range of 0.3% to 14% by weight of the composition and is selected from lactose monohydrate, silicified microcrystalline cellulose, cross carmellose sodium, hydroxyl beta cyclodextrin, polysorbate 80, colloidal silicon dioxide, hydroxypropyl methyl cellulose or a combination thereof.

12. The composition as claimed in claim 1, wherein the film coating is a mixture of coating premix, sodium lauryl sulphate and water.

13. (canceled)

14. (canceled)

15. (canceled)

16. (canceled)

17. The pharmaceutical composition as claimed in claim 1, comprising the compound of Formula IV along with a solubilizing agent; wherein the compound of Formula IV is selected from a group comprising Formula V

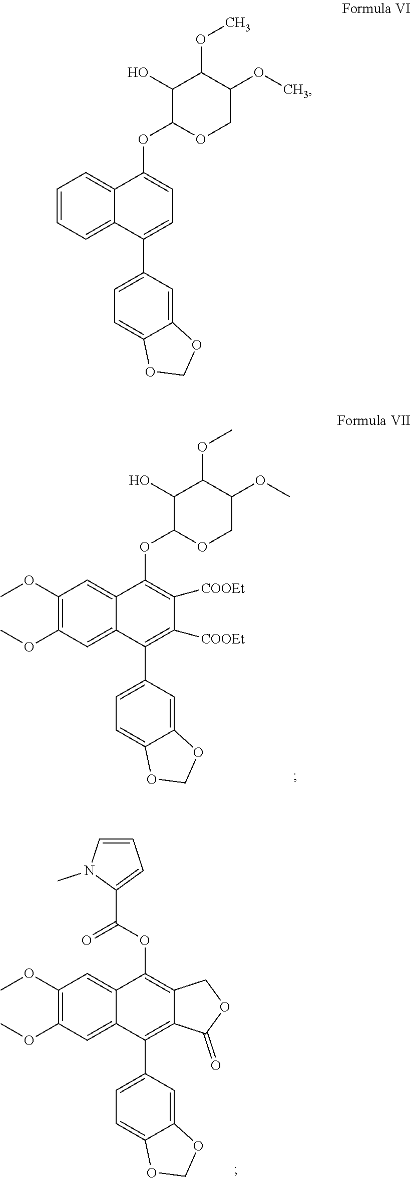

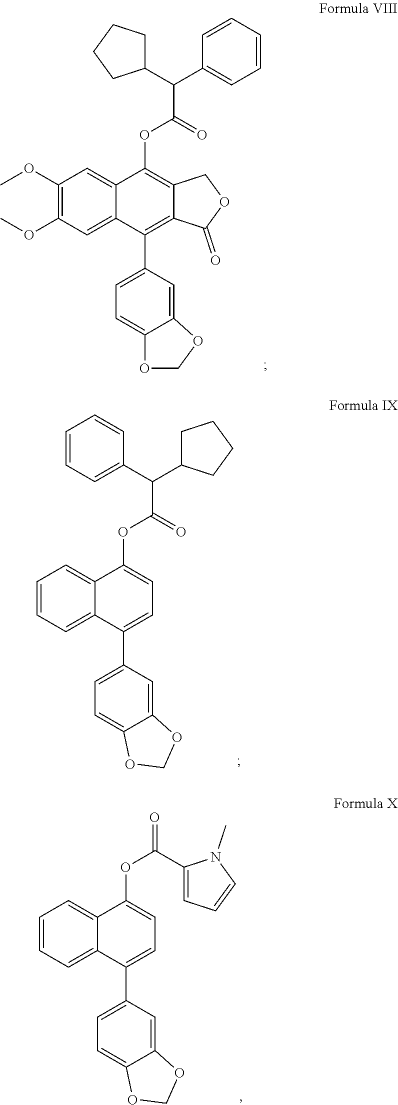 [SMILES: COc1cc2c(OC(=O)C(c3ccccc3)C3CCCC3)c3c(c(-c4ccc5c(c4)OCO5)c2cc1OC)C(=O)OC3 ; O=C(Oc1ccc(-c2ccc3c(c2)OCO3)c2ccccc12)C(c1ccccc1)C1CCCC1 ; Cn1cccc1C(=O)Oc1ccc(-c2ccc3c(c2)OCO3)c2ccccc12]

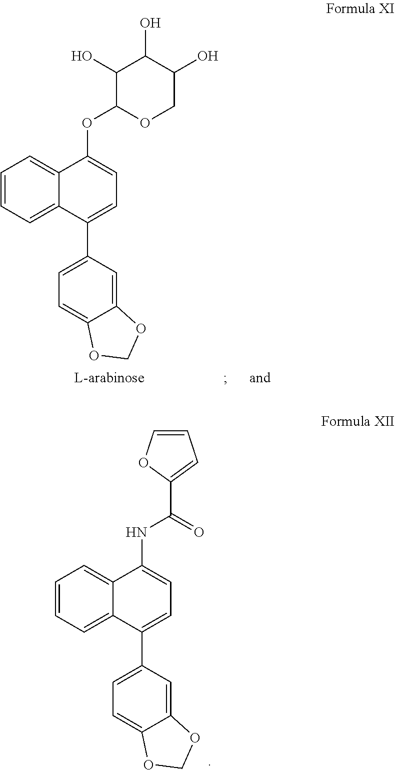 [SMILES: OC1COC(Oc2ccc(-c3ccc4c(c3)OCO4)c3ccccc23)C(O)C1O ; O=C(Nc1ccc(-c2ccc3c(c2)OCO3)c2ccccc12)c1ccco1]

18.-24. (canceled)

25. The composition as claimed in claim 1, wherein it is in a dosage form suitable for oral administration.

26. The composition as claimed in claim 25, wherein the dosage form is a tablet.

27. A method of preparing the pharmaceutical composition as claimed in claim 1, by hot melt extrusion wherein the solubilizing agent is polyvinylpyrrolidone.

28. (canceled)

29. A method of treating cancer or cancer related diseases in an individual, comprising administering to the individual an effective amount of the pharmaceutical composition comprising any of the compound of Formula IV along with a solubilizing agent.
